(19) United States
(12) Patent Application Publication
(10) Pub. No.: US 2009/0073717 A1
(43) Pub. Date: Mar. 19, 2009

(54) LIGHT SOURCE TRANSMITTING ASSEMBLY OF HAND-HELD MEDICAL ILLUMINATING DEVICE

(76) Inventor: Donny CHUNG, Taipei (TW)

Correspondence Address: HDLS Patent & Trademark Services, P.O. BOX 220746, CHANTILLY, VA, 20153-0746, US

(21) Appl. No.: 11/856,795

(22) Filed: Sep. 18, 2007

(51) Int. Cl. A61B 1/06

(52) U.S. Cl. 362/572

(57) ABSTRACT

A light source transmitting assembly of a hand-held medical illuminating device includes an elongated light source transmitting body, a protective body and a connecting body. The light source transmitting body is constituted of an optical fiber and a covering body made of a flexible metallic material. The protective body is provided thereon with a conical body. The front end of the conical body has a curved surface and the rear end thereof is provided with a connecting hole. The connecting hole is connected to one end of the light source transmitting body. The connecting body is provided therein with a penetrating hole that allows the other end of the light source transmitting body to be inserted therein. The connecting body can be assembled with a grip of the hand-held medical illuminating device. The covering body prevents the light from leaking during the transmission of the light source. The flexibility of the covering body can allow the illuminating angle of the light source transmitting body to be adjusted.

### BACKGROUND OF THE INVENTION

1. Field of the Invention

The present invention relates to a light source transmitting assembly of an illuminating device, and in particular to a light source transmitting assembly of a hand-held illumination device.

2. Description of Prior Art

Medical instruments have to be combined with some advanced technologies such as micro mechanical and electrical technology, communication technology, material technology and bio-chemical technology to form another industrial technology. Therefore, the development of each kind of technique or technology can promote the industry of medical instruments. Owing to the improved medical instruments, the possibility of the success in the medical treatment can be also increased.

The illuminating device used in a traditional medical treatment can be mounted on a ceiling or wall (such as an operation lamp), and alternatively, can be attached to the head of medical personnel. When the medical personnel perform a medical treatment for a patient, the illuminating device is used to illuminate the position of the wound on the body of the patient, so that the medical personnel can see the wound of the patient clearly. In addition to the above two kinds of illuminating devices, another kind of illuminating device currently used by the medical personnel is a hand-held illuminating device. The volume of such kind of hand-held illuminating device is small, so that it can be used to illuminate the wound of the patient if the above two kinds of illuminating devices cannot work in some instances.

However, as shown in FIG. 1, the conventional hand-held medical illuminating device has a grip 10. The interior of the grip 10 is provided with a battery 20, and the front end thereof is assembled with a tube body 30. The front end of the tube body 30 is provided therein with a light-emitting source 40. The light-emitting source 40 is electrically connected with a switch 60 and the battery 20 via electricity-delivering leads 50. In use, the medical personnel only presses the switch 60, the electricity is delivered from the battery 20 to the light-emitting source 40 via the electricity-delivering leads 50, thereby lighting up the light-emitting source 40. In this way, the medical personnel can see the wound of the patient clearly. Since the tube body 30 of the conventional hand-held medical illuminating device is made of a hard metallic material, it is not easy for the medical personnel to adjust the angle or orientation of the illuminating device. Therefore, the light emitting by the illuminating device may be obstructed by some mussel tissue, which results in a fact that the side or corner of the wound of the patient may not be seen and thus generates a trouble for the medical personnel in performing the medical treatment.

### SUMMARY OF THE INVENTION

In view of the above drawbacks, the present invention provides a light source transmitting assembly of a hand-held medical illuminating device, in which the angle or illuminating orientation thereof can be adjusted. With this arrangement, the medical personnel can see every corner of the wound of the patient clearly.

The present invention is to provide a light source transmitting assembly of a hand-held medical illuminating device, which includes a light source transmitting body, a protective body and a connecting body. The light source transmitting body is constituted of an optical fiber and a covering body. The protective body is provided thereon with a conical body. The front end of the conical body has a curved surface and the rear end thereof is provided with a connecting hole. The connecting hole is connected to one end of the light source transmitting body. The connecting body is provided therein with a penetrating hole that allows the other end of the light source transmitting body to be inserted therein. The connecting body can be assembled with a hand-held grip.

### BRIEF DESCRIPTION OF THE DRAWINGS

FIG. 1 is a schematic view showing a hand-held mechanical illuminating device of prior art;

FIG. 2 is a perspective view showing the structure of the light source transmitting assembly of the present invention;

FIG. 3 is an exploded view showing the light source transmitting assembly of the present invention;

FIG. 4 is a partially enlarged view of FIG. 3;

FIG. 5 is a cross-sectional view showing the light source transmitting assembly of the present invention; and

FIG. 6 is a schematic view showing an exemplary application of the light source transmitting assembly of the present invention.

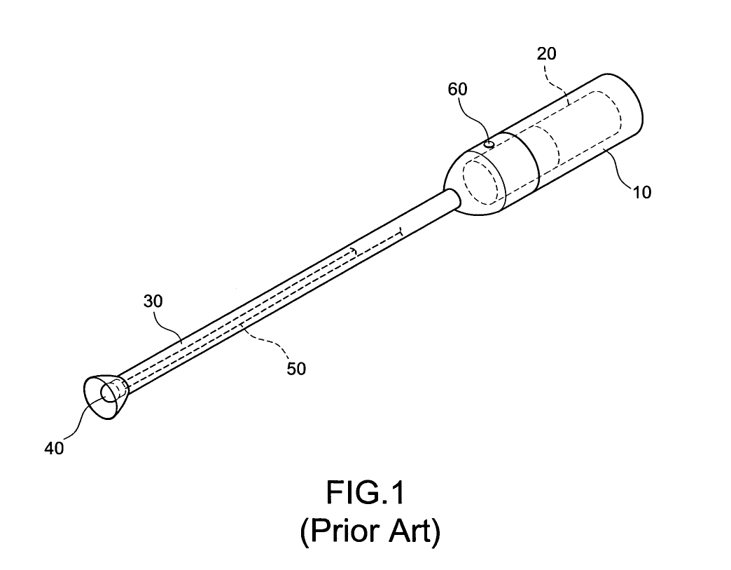

### DETAILED DESCRIPTION OF THE INVENTION

The technical contents and the detailed description of the present invention will be explained with reference to the accompanying drawings.

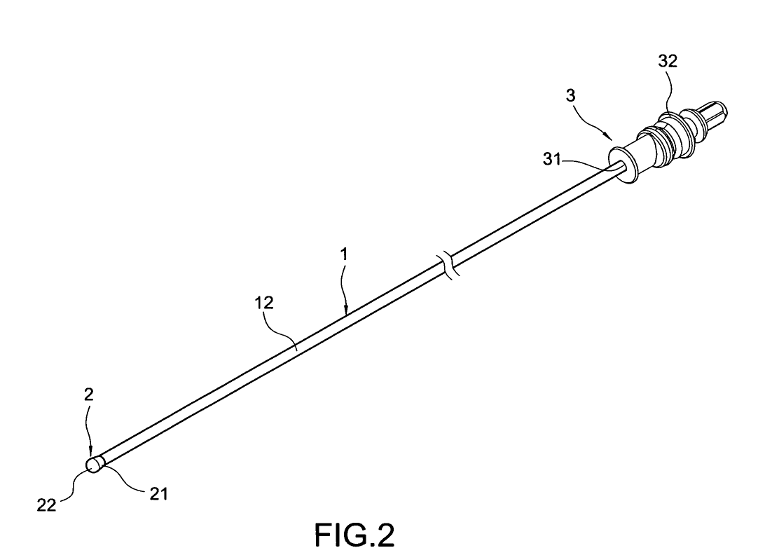

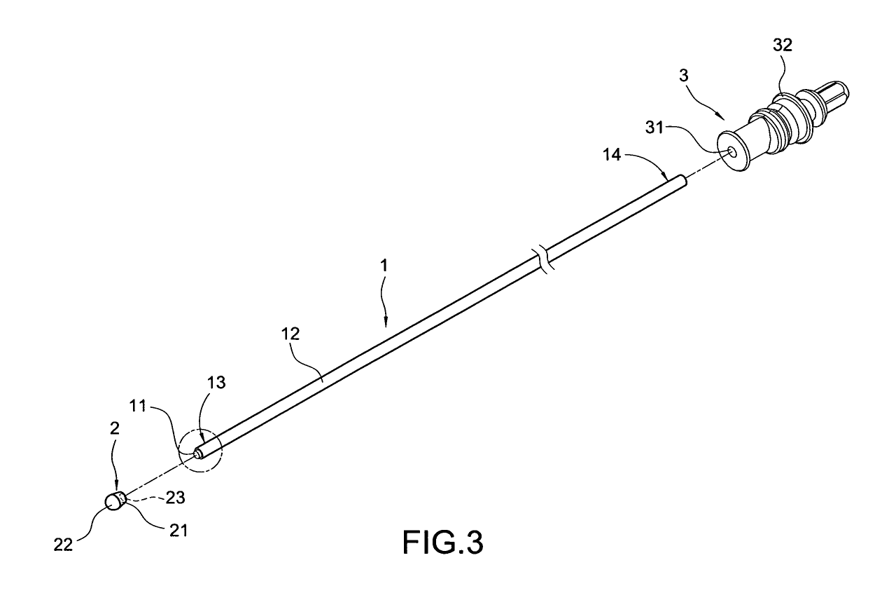

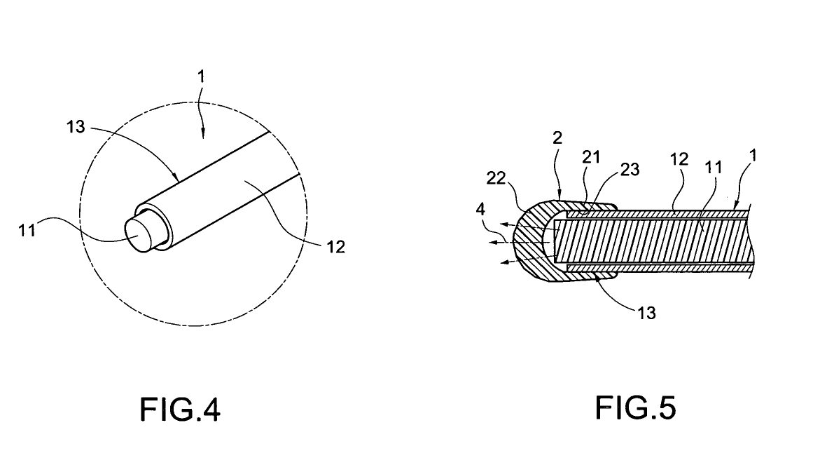

FIG. 2 is a perspective view showing the structure of the light source transmitting assembly of the present invention. FIG. 3 is an exploded view showing the light source transmitting assembly of the present invention. FIG. 4 is a partially enlarged view of FIG. 3. These figures show a light source transmitting assembly of a hand-held medical illuminating device. The light source illuminating assembly includes an elongated light source transmitting body 1, a protective body 2 assembled with an end of the light source transmitting body 1, and a connecting body 3 assembled with another end of the light source transmitting body 1.

The light source transmitting body 1 is constituted of a linear transparent optical fiber 11 and a covering body 12 covering on the outer surface of the optical fiber 11 (as shown in FIG. 3). The light source transmitting body 1 is provided thereon with a first end 13 and a second end 14. The light source transmitting body 1 is used to transmit a light source (light source generating end) from the second end 14 to the first end 13, thereby providing an illumination. The covering body 12 prevents the light from leaking during the transmission of the light source. The covering body 12 is made of a flexible metallic material. The covering body 12 covers on the optical fiber 11, thereby adjusting the illuminating direction of the optical fiber.

The protective body 2 is made of a transparent material and has a conical body 21 thereon. The front end of the conical body 21 is provided with a curved surface 22, and the rear end thereof is provided with a connecting hole 23. The connecting hole 23 is used to connect on the first end 13 of the light source transmitting body 1. The protective body 2 can prevent the sharp corner of the first end 13 from hurting the wound of the patient. Further, the protective body 2 is used to condense or scatter the light transmitted by the light source transmitting body 1 onto a predetermined position.

The connecting body 3 is provided therein with a penetrating hole 31 that allows the second end 14 of the light source transmitting body 1 to be inserted therein. The connecting body 3 is provided thereon with a projecting ring 32. When the connecting body 3 is assembled with a hand-held grip (not shown), the projecting ring 32 blocks the front end of a connecting tube of the grip.

With reference to FIG. 5, it is a cross-sectional view showing the light source transmitting assembly of the present invention. When the light source transmitting assembly of the present invention is assembled with a hand-held grip (not shown), the light 4 of the light source generating end within the hand-held grip is transmitted via the optical fiber 11 of the light source transmitting body 1. When the light 4 is transmitted to the protective body 2, the protective body 2 can condense or scatter the light 4. The light 4 can be refracted to the predetermined position via the curved surface 21 of the protective body 2. Further, when the light source transmitting body 1 is used to illuminate the wound of a patient, the protective body 2 can prevent the sharp corner of the first end 13 from hurting the wound of the patient.

When the light 4 is transmitted in the optical fiber 11, the covering body 12 covering on the outer surface of the optical fiber 11 can prevent the light 4 transmitted by the optical fiber 11 from leaking, so that the light 4 can be transmitted to the protective body 2 efficiently.

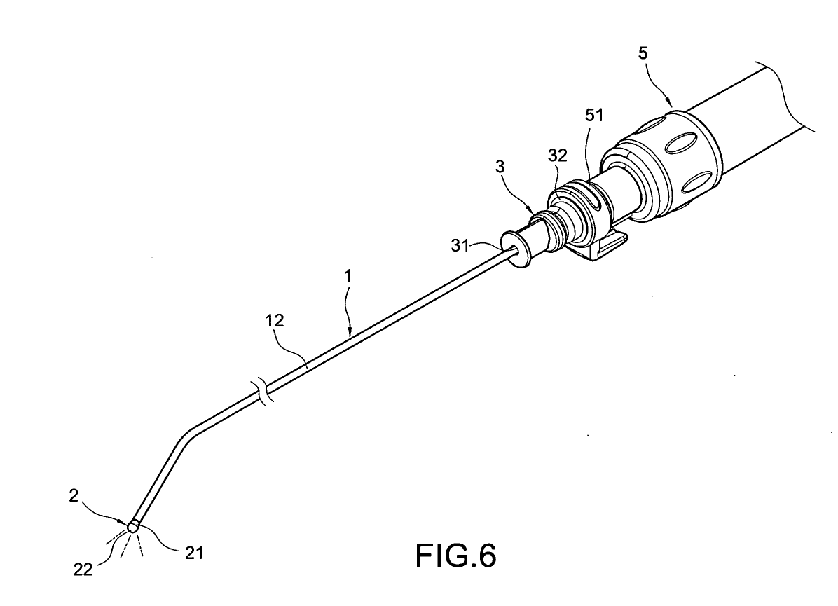

With reference to FIG. 6, it is a schematic view showing an exemplary application of the light source transmitting assembly of the present invention. As shown in this figure, when the light source transmitting assembly is assembled with a hand-held grip 5, the connecting body 3 is inserted into a connecting tube 51 at the front end of the hand-held grip 5. At this time, the projecting ring 32 can block at the opening of the connecting tube 51.

In use, the medical personnel can hold the hand-held grip 5 directly by his/her hand. A battery (not shown) within the hand-held grip 5 lights up a light-emitting body (not shown and can be a bulb or light-emitting diode), the light 4 can be transmitted via the light source transmitting body 1. When the light 4 is transmitted to the protective body 2, the light 4 can be condensed or scattered, and is refracted onto the predetermined position via the curved surface 21 of the protective body 2. In use, if the light projected by the protective body 2 cannot illuminate a certain position of the patient body, the flexible metallic covering body 12 can be used to adjust the angle of illumination, thereby illuminating the predetermined position.

Although the present invention has been described with reference to the foregoing preferred embodiment, it will be understood that the invention is not limited to the details thereof. Various equivalent variations and modifications can still occur to those skilled in this art in view of the teachings of the present invention. Thus, all such variations and equivalent modifications are also embraced within the scope of the invention as defined in the appended claims.

What is claimed is:

1. A light source transmitting assembly of a hand-held medical illuminating device, comprising:
a light source transmitting body including an optical fiber and a covering body covering on an outer surface of the optical fiber, the light source transmitting body being provided thereon with a first end and a second end; and
a protective body assembled on the first end of the light source transmitting body.

2. The light source transmitting assembly of a hand-held medical illuminating device according to claim 1, wherein the optical fiber is a linear transparent body.

3. The light source transmitting assembly of a hand-held medical illuminating device according to claim 1, wherein the covering body is made of a flexible metallic material.

4. The light source transmitting assembly of a hand-held medical illuminating device according to claim 1, wherein the protective body is made of a transparent material and has a main body thereon, a front end of the main body has a curved surface and a rear end thereof is provided with a connecting hole.

5. The light source transmitting assembly of a hand-held medical illuminating device according to claim 4, wherein the main body of the protective body is formed into a conical shape.

6. The light source transmitting assembly of a hand-held medical illuminating device according to claim 1, wherein a connecting body is connected onto the second end of the light source transmitting body, the connecting body is provided therein with a penetrating hole, and an outer surface of the connecting body is provided with a projecting ring.
